(19) United States
(12) Patent Application Publication
(10) Pub. No.: US 2004/0102717 A1
(43) Pub. Date: May 27, 2004

(54) Disposable automatic safety assembly means for test and/or delivery

(76) Inventor: Yan Qi, Highlands Ranch, CO (US)

Correspondence Address: Yan Qi, 421 E. Bexley Lane, Highlands Ranch, CO, 80126, US

(21) Appl. No.: 10/304,609

(22) Filed: Nov. 26, 2002

(51) Int. Cl. A61B 5/00

(52) U.S. Cl. 600/583

(57) ABSTRACT

A disposable assembly within an extendable/retractable punching means addresses the problems of safety and convenience in needle techniques. The assembly enables a preset application in such assembly. The punching means comprises a punching needle and a recoverable pad. A housing pod of the assembly houses the needle and the recoverable pad. The needle is able to extend from the housing pod to puncture a subject and retract within the housing pod after puncturing. The assembly contacts and/or adheres to the subject when it is used. It may do puncture the needle, performance the preset application, and conceal the needle in one operating motion. The invention assembly avoids any potential risks associated with needle techniques: using no sterile needle, needle-stick, needle irritation, contamination and cross-infection. The assembly is of simple mechanism to easily make with economic benefits and to conveniently perform with fewer malfunctions.

### BACKGROUND OF THE INVENTION

Due to increasing health risks in the areas of healthcare that includes medicine and biotechnology industry, a higher degree of awareness has arisen concerning problems associated with needle techniques. For example, occupational risks in the clinical setting for infectious agents such as hepatitis, HIV, and other pathogens have generated a demand for safer techniques. Usually, current methods for withdrawal of sample or delivery of pharmacological agents require a high degree of professional training. But these remain high risk of needle-stick and contamination in the trained person. However, with homecare becoming more prevalent in developed countries, the demands for safer and more convenient techniques will be even more important in the future. Patented technologies have been developed to address the problems inherent in current withdrawal/delivery techniques. However, the designs of devices developed to date do not fully address safety and convenience issues.

In prior patents, safer devices that involve retractable syringes or lancing devices have been developed in order to address the issue of accidental needle-sticks and easy use. In view of such situation, various systems have been developed.

In delivery systems, there are a lot developed retractable syringes. Because the injection needle of these syringes is an exposed needle in using process, it also carries risks associated with accidental needle-sticks. The retractable syringes have two units that one is for inserting needle (it may be also for retracting) and other for delivery. These devices are used more than one operation motion to perform the filled drug, e.g. inserting the needle, sending drug, retracting the needle and so on. They are operated with the complex steps and with complex mechanism. Because the devices are difficulty to perform the delivery without a piston, they are kinds of piston driver systems.

In testing systems, the safety devices, wherein a needle (lancet) and testing meter are integrated, usually are disclosed for monitoring glucose. For example, in U.S. Pat. Nos. 4,787,398, 6,183,489, 6,315,738, 6,352,514, and 6,379,317, these devices usually house a puncture needle, which is held by a needle holder to connection a puncture driver. However, these devices still use an apparatus with an exposed needle, and there remains a certain level of risk for accidental needle-sticks.

The previously mentioned devices may have shortcomings regarding safety and convenience. That is, these devices involve an exposed needle during the puncture procedure. The puncture procedure includes that before the insertion, or during the process of insertion, or after the insertion. The users need to deal with the exposed needle and a non-protecting puncture site. These devices are problematic for at least one of follows: 1. The exposed needle may cause accidental needle-sticks to users, especially non-professed person. 2. The exposed needle does not warranted to use a sterile needle. 3. The blood or body fluid from the punctured site of the subject may become a source of contamination and cross-infection. These current devices do not available to protect the punctured site immediately. 4. In some devices, the house or sleeve of the devices pushed down against the puncture site of the subject, e.g. the skin, it is repeatedly used. The house or sleeve is the source of contamination and cross-infection. 5. In the testing system, these current devices do not warranted to take enough samples from the punctured site of the subject. Especially, the punctured site of the subject is other than a fingertip, such as limbs.

Furthermore, These devices do not fit the requirements for simple and convenient procedure. These need more than one motions to perform testing or delivery. For example, The current testing devices use complex steps: like loading a needle holder, exposing a sharp of lancet, loading a test component, moving the test component to take sample from punctured site, disconnecting the loaded item, covering the sharp end of the puncture needle, and so on. The current retract delivery syringes use complex steps: like exposing a sharp of needle, puncturing the needle, delivering filled drug into the subject, retracting the needle back, taking a bandage to cover the punctured site and so on.

In view of the situation, there is a strong demand for an assembly that provides automatic safety and convenience issues.

### SUMMARY OF THE INVENTION

In view of the foregoing, an object of the present invention is to provide a healthcare assembly, which addresses the problems of safety and convenience in needle techniques. The present invention is of a simple mechanism that easily be made with economic benefits and easily performs with fewer malfunctions.

This and other objects of the present invention are achieved by providing an assembly with an extendable/retractable punching means for safety and convenience performance the testing and/or delivery task.

In one embodiment, the present invention provides a disposable assembly with an extendable/retractable punching means to maintain the needle in sterilized condition before use. The present invention also provides a sharp free environment to user. The extendable/retractable punching means comprises a punching needle and a recoverable pad. The punching needle and the recoverable pad are disposed in the pod of assembly. The sharp end of the needle may be concealed in the recoverable pad for maintaining sterile. The recoverable pad is pressed by the needle to change its shape, so the needle is able to run out from the assembly to puncture the subject. The needle is able to automatic retract back via the recovery power of the recoverable pad which frees from pressure.

The extending/retracting ability of the needle allows the sharp end of the needle out the assembly into the subject only during puncture, and allows the sharp end of the needle to be protected within the assembly before and after puncture. Then the needle is automatically and silently retracted back into the assembly after puncture, the assembly prevents continued trauma and contamination in relation with the needle.

In one embodiment, the present invention provides a disposable assembly with an extendable/retractable punching means and at least one preset application. The disposable assembly touches against the target subject surface before puncture, punctures the needle into the subject, and performs the preset application task, retracts the needle automatically back to a safe position after puncture. The application means an application container within applied components for testing or delivery task. The task performance of the disposable assembly follows the needle extension. Further more, the recoverable pad may conceal the needle in sterile condition before use, may store the preset applied container, may provide a delivery power to send the filled injection drugs into the subject, and may store vacuum to take enough samples from the subject.

In one suitable approach, the present invention may provide one test container in the assembly for test task. The test container houses test component (e.g. strip or chip) that measures the caught sample from the subject. The testing assembly may be used one motion to puncture the lancet needle, to perform the preset test application, and to retract the needle after puncture. In this case, the assembly may be able to perform the entire test process directly on the surface of subject.

In one suitable approach, the present invention may provide one delivery container in the assembly for delivery task. The delivery container is filled with injectable substance to be delivered into the subject. The delivery assembly may be used one motion to insert the hollow needle, to deliver the substance into the subject, and to automatic retract the needle. Specifically, the assembly may be a piston free delivery system to perform the delivery task.

In one suitable approach, the present invention may provide suction to the puncture site. The suction means vacuum power that already stored in the assembly. So the present invention may provide the assembly ensure sufficient sample, e.g. body fluid, blood, from the punctured site for the preset electrochemical or optical measuring system.

In one suitable approach, the present invention may have a dressing portion that is in contact with the subject's surface. The assembly may dress the surface of the target subject to provide protection to the puncture site. The wafer assembly may be a thin and lightweight article, like a bandage, that conveniently dresses the puncture site.

In one embodiment, the present invention may be easily combined with an associated device. The device may be as a tool for easily performing the assembly. The device with an operating mechanism and/or a measuring mechanism includes one of follow device: a testing device, a lancing device, a scanning device, and an injection device. The assembly may be load to the device and performed by the device.

In one suitable approach, the present invention may enable input and output of applicable information to and from the associated device via an Internet interface.

In one embodiment, the present invention may provide disposable assemblies to a completely automated system for performing the task of the assembly. In this case, the assembly may be able to perform its function automatically and without further human action after being triggered.

### BRIEF DESCRIPTION OF THE DRAWINGS

FIG. 1A is a sectional view of one assembled embodiment having a cylinder pod.

FIG. 1B is a sectional view of cylinder pod's assembled embodiment loaded to an associated device.

FIG. 2A is a sectional view of one assembled embodiment having a wafer pod with a delivery container.

FIG. 2B is a sectional view of wafer pod's assembled embodiment loaded to an associated device

FIG. 3 is sectional view of assemblies with the different recoverable pads and the different needles and others. FIG. 3 includes 3A, 3B, 3C, 3D, 3E, 3F, 3G, and 3H.

FIG. 4A is a sectional view of one assembled embodiment with a delivery container in the recoverable pad.

FIG. 4B is a sectional view of one assembled embodiment with two-piece pad for a delivery container.

FIG. 4C is a sectional view of one embodiment with a delivery container in the needle-head.

FIG. 4D is a sectional view of one assembled embodiment with a delivery container in the needle-head with a plunger.

FIG. 5A is an exploded perspective view of one embodiment with a test container.

FIG. 5B is a sectional view of one assembled embodiment of assembly with a test container.

FIG. 5C is a sectional view of one assembled embodiment of assembly with a test container stand in the pod.

FIG. 5D is a sectional view of one assembled embodiment of assembly with a slide test container.

FIG. 5E is a sectional view of one assembled embodiment of assembly with a test container loaded to an associated device.

### DETAILED DESCRIPTION OF THE INVENTION

In order to better understand the invention, the following detailed descriptions of its parts and functions are given. The following preferred embodiments are described with drawings, with the understanding that these drawings depict only construct embodiments of the invention and are not limited to the scope.

The present invention is an automatic safety assembly 100 within an extendable/retractable punching means. A needle combined with a recoverable pad forms an extendable/retractable punching means for the assembly. The construct of the inventive assembly is of a simple mechanism, which reduces the number of malfunctions and operating steps. The assembly not only easily allows the needle to remain sterile after be sterilized, but also conveniently allows the sharp free (the sharp of needle free) environment in the performance. It would avoid any risk of accidental needle-stick, and any risk of biological contamination.

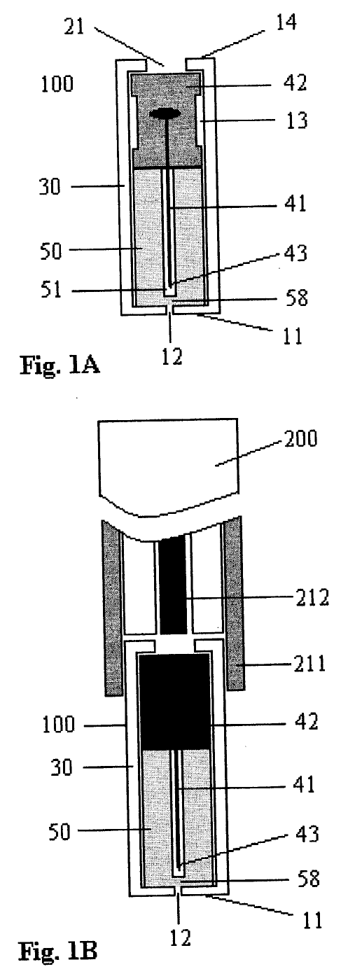

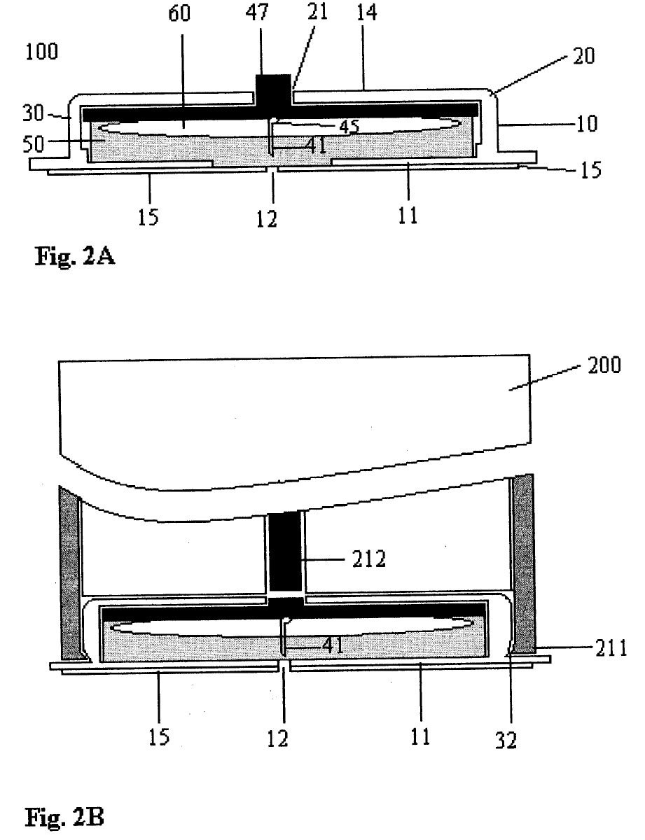

In one embodiment, the present invention may provide a disposable automatic safety assembly that contact against surface of the target subject, e.g. the skin of a patient. Referring FIGS. 1-2, the assembly has a housing pod and an extendable/retractable punching means for healthcare. A pod 30 is one integrated piece to house the extendable/retractable punching means. The pod 3b may be a cylinder piece (FIG. 1) or a wafer piece (FIG. 2). The pod is not limited to the shown shapes; it includes any shape pod, e.g. cube, rectangular cube, etc. The Pod 30 may comprise a base member 10 and a top member 20. The pod is not limited to the two members, it may be one member (only a pod) or more than two members. The pod 30 has two major surfaces, bottom-surface 11 and top-surface 14. Usually the bottom-surface 11 is at the base member 10 and the top-surface 14 is at the top member 20.

The pod 30 has a house 13 for setting the extendable/retractable punching means. The extendable/retractable punching means comprises a punching needle 40 and a recoverable pad 50. The punching needle 40 usually sits on the recoverable pad 50.

The punching needle 40 may be a “tack” shaped needle, which may comprise a needle-body 41 with a sharp end 43 and a needle-head 42. The recoverable pad 50 provides a support to the needle 40 via the needle-head 42 contacted to it. In the present invention the punch needle may be fused or stuck on the recoverable pad 50 as one piece.

The recoverable pad 50 is made of flexible materials. For example, the flexible material is foam rubber. After the needle 40 and the pad 50 are set into the house of pod 13, the needle-body 41 may be in a vertical position relative to the pod 30. The needle 40 being moved up or down, it is dependent on the changes of the recoverable pad 50 that change its shape and/or position. Prior to puncture, the needle 40 is located at a position of the pod called initial position. The initial position means that the needle 40 usually touches the upper surface of the recoverable pad 50 or lightly presses the recoverable pad 50. Because the needle-body 41 may be a bit shorter than the thickness of the recoverable pad 50, the needle-body is concealed into the recoverable pad 50 without the sharp end 43 being exposed. For puncture into the subject, the needle is pressed or punched on the needle-head 42 by a pressing or punching power. The needle-body 41 is moved in the recoverable pad toward the direction of the subject to extend out the pod 30 into the subject when the recoverable pad 50 is pressed down by the needle-head 42. The needle 40 would be automatically retracted back into the pod via the recovering power of the pad 50 when the pressing or punching power is withdrawn. The size and shape of the needle-head 42 may match the size and shape of the house 13, so the needle goes straight in the house of pod to guide the needle's direction up or down.

The base member 10 of the pod may have a hole 12 that acts as a pathway for the needle to puncture into the subject. The base member maybe has a dressing portion 15 at its bottom-surface 11 that may adhere to the subject's surface. The lower surface of recoverable pad 50 may also have a dressing portion 15 when the recoverable pad 50 is exposed and touched to the subject.

In the case, the assembly has a wafer pod, which enables to be a thin and lightweight article (FIG. 2). The wafer assembly, like a bandage, may be easily adhered to the subject surface. If the assembly remains on the subject, it is usually stuck on the surface of subject to protect the puncture site temporarily. The protection of the puncture site is of benefits for avoiding contamination at the puncture site. As for the dressing portion 15, the portion is not only made of the material for adhesion to the subject, but also made of anesthetic and/or antibiotic reagents. Said anesthetic reagent means for reducing the pain in the puncturing and said antibiotic reagent means for reducing the risk of infection at the puncture site.

Further, there may be a shallow space on the bottom-surface of the pod. The shallow space contributes a feature that user never touches the area of the assembly where is a path of needle running.

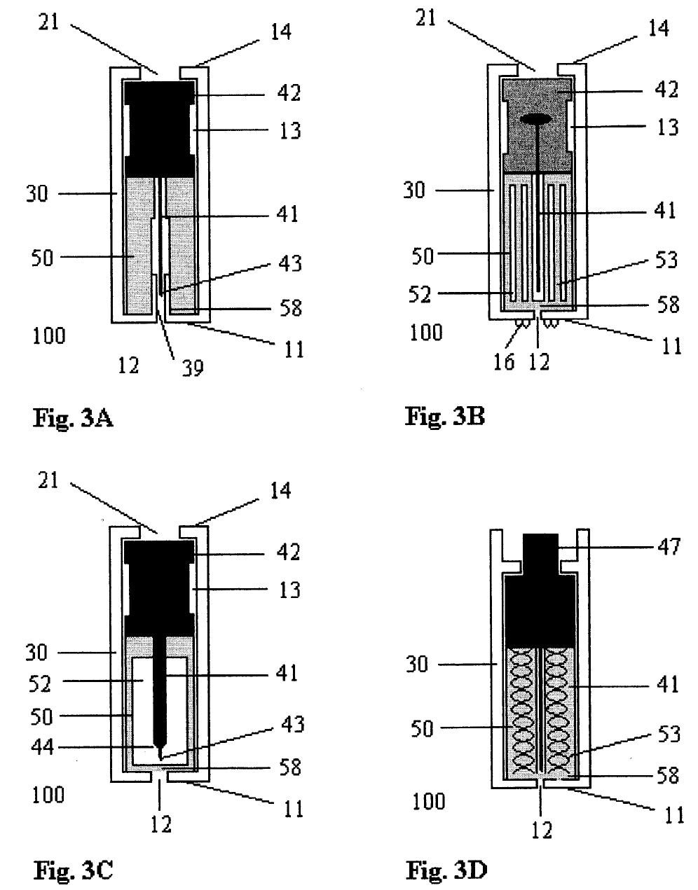

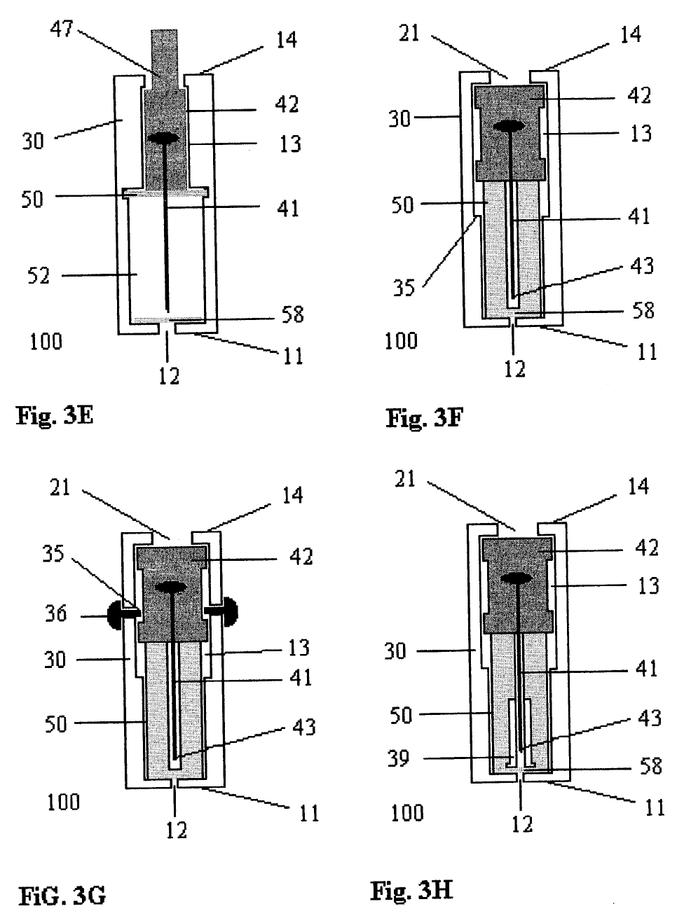

Further, the base member may have a ring member 39 to be connected to the hole 12. The ring member 39 is located in the house of pod 13 (shown on FIG. 3A). Said ring member 39 may be over the bottom-surface of the pod 11. The ring member 39 is around the needle-body 41, and may define the guidance of the direction of needle. It is especially useful for insertion of a fine needle into the subject. In testing application, the ring member has more functions than the needle guide (detail in testing part).

The top-surface 14 of the pod has an opening 21, which may be used to actuate said extension of needle. Usually the top member 20 of the pod has the opening 21 on it. Performing the assembly, the needle is pushed on the needle-head 42. In some cases, the needle-head 42 may not be expose to the outside of the pod. For example, the punching power runs through the opening 21 to press on the needle-head. In some cases, the needle-head 42 may have a protrusion 47. The protrusion of needle-head 47 may extend beyond the opening 21 (shown on FIGS. 3D, 3E, 2A).

In general, there are two options to perform the assembly. The first option is performed by a perform-member 212 of an associated device 200. The assembly may have a connector-site 32 on the pod 30. The connector-site 32 enables the assembly to be loaded to the associated device 200. The associated device has at least one couple-connector 211 and a perform-member 212. The couple-connector 211 uses to connect the connector-site of the assembly 32 and load the assembly. The perform-member 212 is a punching member or pushing member (e.g. a mini-hammer like) to perform the loaded assembly. The perform-member 212 usually runs through the opening 21 to punch the needle-head 42. But, when the protrusion of needle-head 47 extends beyond the opening 21, said perform-member 212 directly punches the protrusion 47 (shown on FIG. 3D). In the present invention, the perform-member 212 not needs connection to the needle-head. This makes the associated device convenient loading and using the assembly.

The second option is performed by user's hand manually pushing the protrusion of needle-head 47 directly.

In the invention, the depth of puncture depends on the distance of the needle 40 down. The depth of puncture may be a pre-set depth before performing the assembly. It is pre-set by the associated device or by the assembly itself. The associated device sets the pre-set depth via setting the moving distance of its perform-member 212. The assembly may already have a pre-set depth for the needle. There is a blocker 35 in the house of the pod (FIG. 3F). The blocker 35 stops the needle-head 42 going down. If the blocker 35 is movable member that is part of a changer-member 36, the assembly may change a pre-set depth by turning the change-member 36. This means the change-member 36 moves on outside of the pod 30 to change the position of blocker member in the house 13 (FIG. 3G). When the change-member moves the blocker 35 to lock the needle-head at the initial position, the assembly is in the safe status that can not be actuated.

An application container for a preset application task is integrated in the inventive assembly. The application container is configured for a test or delivery task. The application container includes both an open application container and a closed application container. In the invention, the application container has variety locations of setting in the assembly. The application container may set in one of following location: the inside of the pod, the inside of the pad, the inside of the needle or the house of the pod. The application container of the assembly houses applied components. The application container filling with the injectable substance, it is called a delivery container 60. And the application container housing test component, it is called a test container 70. The inventive integrated assembly has an ability to perform the preset applied task after the needle 40 is actuated.

Depending on the different preset application in the assembly, both the needle 40 and recoverable pad 50 have a variety of features to fit the different purposes. The needle 40 and recoverable pad 50 may be variant in size for different subjects and different purposes.

According to the recoverable pad 50, the recoverable pad 50 may have a lot of features that contribute to the assembly. First, it may conceal the needle 40 and keeps the needle in sterile condition before use. Second, it is of recovery ability for the needle automatic retracted after being performed. Third, it may provide a space to store the preset application. For example, the recoverable pad stores injectable drugs for delivery application. Fourth, it may provide a delivery power to send the filled injection drugs into the subject. Fifth, it may provide an assistant function to the assembly. For example 1, the pad may have vacuum stored inside to take enough samples for test task. For example 2, the pad provides a deriver to send a movable test container to the prefect position for catching specimen.

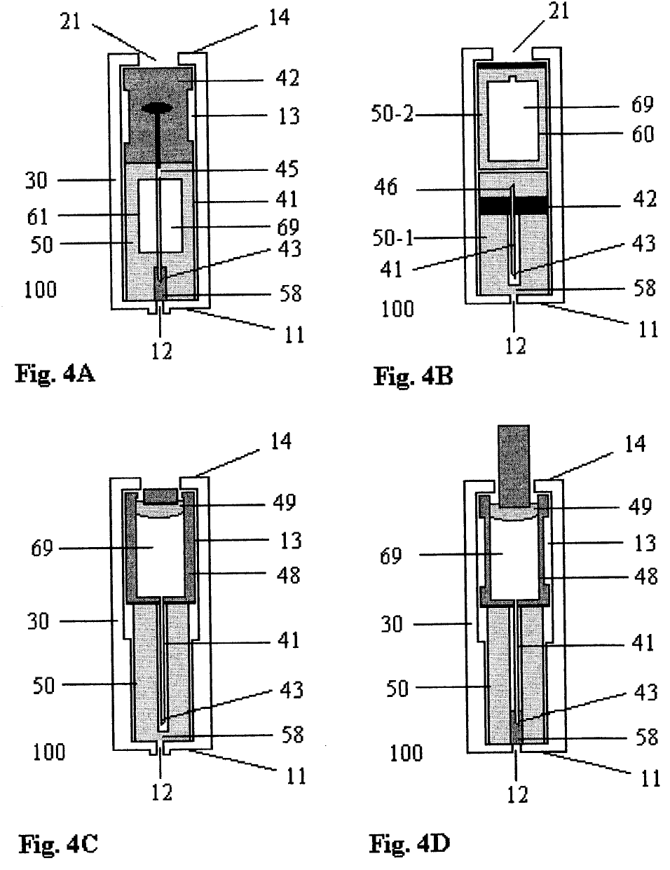

The recoverable pad 50 may be a primary pad, which made of a kind of flexible material, for example, soft rubber or foam rubber. The needle-body may be directly inserted into the primary pad 50 (shown on FIG. 2A). The pad 50 may have a tunnel 51, which is larger than the needle-body 41 to make the needle easily moving in the pad. Usually the tunnel may be not a complete tunnel that hides the needle in the closing pad. This means that a penetrable member 58 closes the tunnel (shown on FIGS. 1A, 3D, 3G). The penetrable member 58 may be either a thin member just to close the end of the tunnel or a thick member around the sharp end of the needle. When the needle is the hollow hypodermic needle, the thick penetrable member is meant to close the hole of the sharp end before using the assembly (shown on FIGS. 4A, 4D, 2A).

In general the pad 50 is made of material with recovery ability. This means that any available material with recovery ability can be the material for the pad. Preferred material is soft material and/or foam material that has springy recovering power to support the needle retracting back. Said foam material means any kind of foamed materials. Said soft material means any soft springy materials.

The recoverable pad may be made of different flexible materials, it is called mixed pad. In the mixed pad, it is possible that there are different recovering powers at the different points of the pad. For example, some areas are made of rubber and some areas are made of foam rubber. In addition, the pad 50 may adopt other firm springy material setting into the pad. The firm springy material may be adopted as a strong recovering power to the mixed pad. Referring FIG. 3D shown springs set inside the mixed pad.

Additionally, the inside of the pad 50 may have a room or rooms 52. The room or rooms would make the pad 50 more flexible. The rooms may be produced by a kind of foamed material, e.g. sponge material, or by inside supports 53. Preferably the supports 53 are vertically located inside the pad (shown on FIG. 3B). But in the invention, the supports 53 are not limited to the vertical supports 53. The supports can be any directions. Said supports 53 include vertical, horizontal, cross, and mixed, etc. The rooms of the recoverable pad may service as a place for the stored vacuum. The rooms of the recoverable pad may be serviced as a delivery container filled with one dose of drug (detail in delivery part).

Further, the pad 50 may made of two pieces of the pads for the assembly. For example 1: the two-piece pad construction, there is one piece pad being a sealed pad (e.g. sponge pad) filled with vacuum or substance and other piece pad being higher density material that as major resource for the punching means. For example 2: the two-piece pad construction, there is a single larger room between the two-piece. The room between of two-piece pad may be supported by firm material e.g. plastic, so the top piece of the pad is the only resource for extendable/retractable punching means.

Further, the pad 50 may be a thin rubber band member to do the described extendable/retractable function. Said thin rubber member is fused on the pod of the assembly (shown on FIG. 3E). Because the needle 40 may be fused to the thin rubber band member, the thin rubber band member is not only under the needle-head but also be may fused at the top of the needle-head.

Further, the ring member 39 may be set inside the recoverable pad 50 (FIG. 3H).

Depending on the purpose of assembly and the construction of the recoverable pad 50. The needle 40 may includes a hollow hypodermic needle, a lancet needle, and a test probe.

Said hollow hypodermic needle usually is a communication to the delivery container 60 filled with injectable substance. Of cause, the hollow hypodermic needle is available used to withdraw sample from the subject for testing. However, the hollow hypodermic needle is communication with the application container. The sharp end 43 is used to insert into the subject. The hole at other end is used to communication the application container. In the invention, the hole of the other end includes as a side-hole 45 on the hollow needle-body and as an end-hole on the second end 46 of a double-ends hollow needle (detail in delivery part).

Said lancet needle means the sharp end of needle 43 being a sharp edge or point. It is used for puncture the subject to pierce the surface of subject (e.g. the skin of patient) for testing task. The lancet needle is used to obtain the sample from the subject, like blood and body fluid from patient. Further, the lancet needle is available with a second blade-needle 44 on the needle-body. The second blade-needle 44 may have cross sharp edges to pierce the thin penetrable member 38. This means that the lancet has two sharps on its needle-body. When the sharp end 43 pierces the subject, the second blade-needle 44 pierces the thin penetrable member of recoverable pad (shown on FIG. 3C). Opened penetrable member 38 releases the stored vacuum to the puncture site.

Said test probe within absorption material has a sharp end. It is inserted into subject to directly take sample from the subject. The test probe usual is integrated with said test container 70 where reacts with the sample from the subject.

In case of delivery application, the present invention may provide a disposable automatic safety assembly integrated with the delivery container 60. The delivery container filled with injectable medical substance 69 (usually, one dose substance). The injectable medical substance includes liquid substance, encapsulation of substance, gel substance, etc. It may be any kind of drugs e.g. pharmaceutical drug, protein drug, DNA or RNA agent, vaccines, biological, biochemical agent and chemical agent. The filled substance 69 is injected out of the assembly into the subject.

One embodiment, the pad 50 is a place of the delivery container 60. The assembly would serve as a piston free extendable/retractable delivery system. So the pad 50 not only conceals the needle keeping in sterile before use and closing the sharp end 43, but also houses the filled substance for delivery. The chamber 61 is either a room that sealed with thin higher density material e.g. soft rubber, or a plastic drug bag set in the pad. If the substance filled in the bag, the bag is communication to the hole 45 of needle. When the needle is pushed down, the needle-body is punctured into the subject via the pad changed. The sharp end 43, which may be closed in the recoverable pad, is open in the subject. Because the delivery container 60 is pressed too, the pressure can be used to deliver the filled substance 69 into the subject.

In one suitable approach, the delivery container located between the low surface of the needle-head and top surface of the pad. The delivery container 60 is filled with injectable substance 69. The hollow needle-body 40 has a side-hole 45 at the junction point of needle-body and needle-head to communication with the sharp end 43 (shown on FIG. 2A). In here, the plastic drug bag may be a ring shape bag that only already opened to the side-hole 45 (not shown).

In other suitable approach, the pad 50 serves as a whole application container 60. The recoverable pad has a room as a chamber 61 of delivery container, which sealed by thin higher density material e.g. soft rubber (Shown on FIGS. 4A, 4B). The chamber is filled with substance in. The pad seals the side-hole 45 of the needle-body before injection. When pad pressed down, the side-hole opens to the chamber 61 to perform delivery.

In other suitable approach, the pad 50 is a two-piece pad. First piece pad is used for concealing the sharps of needle. Said needle for this case is a double-end hollow needle that has the sharp end 43 and second sharp end 46. Second piece pad is used for delivery container filled with substance (shown on FIG. 4B). When the pressing power or punching power pressed to the second piece, second piece moves to the subject and presses the first piece down to expose the sharps of the double-end needle. One sharp 43 is moved to the subject and other sharp 46 is inserted into the chamber of second piece. The pressing power is either from the associated device or from the user's hand. In the hand case, there is an independent plunger in the house of the pod to be used for pushing. In the approach, one-piece pad is also available for concealing the needle and storing the substance (not shown).

One embodiment is that the needle-head 42 is the place for the delivery container. The needle-head 42 is a syringe like article. Referring FIGS. 4C, 4D. The needle-head 42 comprises a barrel 48 and a piston 49. The barrel 48 has a first end and a second end. The first end of the barrel touches the pad 50 and fixes to the hollow hypodermic needle 40. The second end is open end where the piston 49 fits inside. The internal chamber of the needle-head 42 is filled with said substance 69. When the piston 49 is moved into the internal chamber, the filled substance 69 would be injected into the subject.

In one suitable approach, there is a plunger in the barrel of needle-head. The plunger is push down, it is not only to push the needle-head down, but also push the piston 49 down (shown on FIG. 4D).

In one suitable approach, there is a no plunger in the needle-head (shown on FIG. 4C). The piston 49 is pushed by the perform-member of the associated device 200. The perform-member 212 performs to push both the needle-head down and the piston 47 down. If the associated device 200 has the perform-member 212 and a plunger-member 213, the device uses two steps to perform the delivery. The barrel 48 is pushed by the perform-member 212, and then the piston 49 is pushed by the plunger-member 213 to perform the injection (not shown).

In case of test application, one embodiment is that the present invention may provide a disposable automatic safety assembly integrated with a test container 70. The application container comprises means housing a test component for testing task. The disposable assembly contains the extendable/retractable punching means and the measurement test container 70. The test container 70 which houses test component opens to the puncture site. The test container 70 has a test chamber 71, and a duct 72. The inside of the test chamber 71 arranges test component 73. The duct 72 builds a bridge between the puncture site of subject and the test component 73. This means one end of the duct is open to the subject and the other end of the duct is open to the test chamber. So the duct is a pathway for transport the sample from subject to the test chamber 71 and to test component 73. The duct 72 may be a channel path for transporting the sample by micro capillary action, or may be filled with absorption material for running sample to test component by wicking action, or may be tubing for taking sample by vacuum power.

The duct 72 may have a couple of options to take the test sample for the test component 73.

For example 1: A thin opening of duct channel opens to the hole 12 or the ring member 39. The duct between the base member and the test chamber allows the sample from the punctured site, where the needle 40 pierced, to be transported to the test component.

For example 2: The absorption member of the duct contacts the surface of subject to take sample directly from the surface of subject at pierced site.

For example 3: The duct is connected to the hollow hypodermic needle or the duct is the hollow hypodermic needle. So the hollow needle is drawing sample from the subject to the test component via a vacuum power.

For example 4: the duct is connected to the test probe needle or the duct is set into the test probe. So the probe directly takes the sample from the subject to the test component. In here, said the test container 70 usual is integrated into the test probe.

The assembly may allow the needle to punch the subject (e.g. the skin of patient) and to directly collect fluid sample from the punching site of subject without moving the assembly.

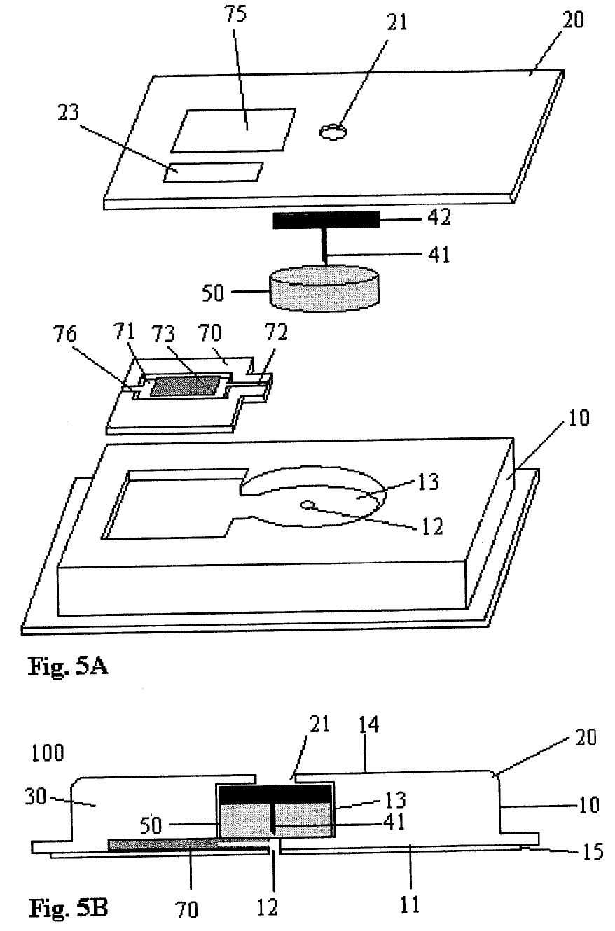

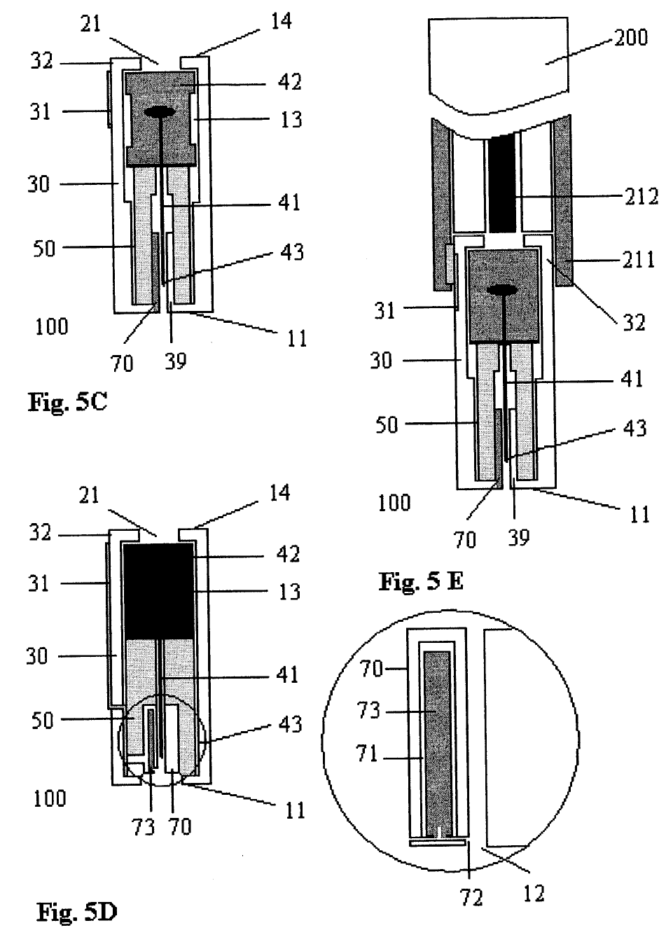

In one embodiment, the test container 70 usually is located in the pod 30. The location of the test container 70 includes that either laid in, or stood in, or angle stood in the pod. Referring FIGS. 5A, 5B and 5C. The test container 70 laid in the base member and stood in the base member. When the test container 70 stood in the pod, it would take some room in the house 13. The test container 70 is availability set very close the needle-body. For example, the ring member 39 is a place for set the test container 70 (FIG. 5C).

After the sample comes into the test chamber 71, the sample reacts with regents of the test component. So most strips, chips, test micro-arrays, and biosensors in current market, which are thin and/or small in size, are available to be integrated into the invention assembly. The test container may be made of firm material e.g. plastic, to protect the test component and avoid damage the construct of the test component. For example, the glucose strip (glucose test component) requires a little of sample for glucose testing (0.3 Ul to 10 ul). The glucose strip or chip is specific fit to be integrated into the assembly.

The test result will be shown to the user by one of the follow suitable approaches.

In one suitable approach, the test container may have a reading window 75 that is a transparent member over the test chamber 71 (shown on FIG. 5A). The result is read through the reading window 75 by an optical instrument of the associated device or by user's eye. In here the test component usually shows a color change after having reacted with the sample.

In other suitable approach, the assembly is loaded to the associated device and transfers electric signal of electrochemical reaction to the associated device. For example, the assembly carrying a blood glucose strip is loaded to a glucose-monitoring meter. It means that electrode members 31 (e.g. electric bars or electrode tabs) on the assembly send electric signal to electrochemical analysis system. The electrode members 31 are located at the connector-site 32 of the assembly and the couple-connector 211 of the associated device. The associated device via the electrode members 31 would receive the electric signal, which is produced from the test component having reacted with the sample.

In other embodiment, the disposable automatic safety assembly integrated with the test container. The test container is a slide test container that slides down in the house of the pod by punching. The slide test container is setting in the lower space of the house of the pod (shown on FIG. 5D). When the pad 50 is pressed to charge its shape for the needle puncture 40, the slid test container is pushed by the pad to be sliding from storing position to the working position. After the pad 50 recovering, the slid test container remains to the working position for testing task. The pod 50 provides a deriver to send the test container to the prefect position for catching specimen.

In other embodiment, the disposable automatic safety assembly integrated with the test container has preset vacuum in the pad 50. The pad has room or rooms to be used for stored vacuum. The second blade 44 on the needle-body pierces the thin penetrable member 58 that follows the sharp end 43 of needle pierces the subject (one of the pad as shown on FIG. 3C). The vacuum power of the pad would suck the sample from the puncture site, so the invention assembly easily takes enough samples for the test task. For avoid the vacuum power leaking, the bottom-surface of the assembly may have a double-ring 16 that tightly is touched the subject (shown on FIG. 3B). The double-ring 16 is made of soft inflexible material. It is located at either bottom-surface of the pod 11 or lower surface of the pad 50.

In other embodiment, the disposable automatic safety assembly integrated with the test container has a path in the pod. The path is used for vacuum power to puncture site, which from outside of source. Usually the path is located between the needle and the house of the pod. So the assembly is loaded to the associated device, the associated device produces vacuum reach to the punch site via the path (not shown).

As description, the assembly not only allows contacting on the subject, puncturing the sterile needle into the subject and retracting the needle to safe position, but also may allow performing the presetting application and protecting the puncture site. The entire process would be finished by one operating motion. The invention assembly provides a safe and convenient article for healthcare. If the assembly integrated with both test container 70 and delivery container 60, the assembly would perform both testing and delivery tasks.

In general, the process of performing this inventive assembly comprises the steps of:

1. Applying the disposable assembly to puncture area of subject, e.g. the skin of patient.

2. Performing the extendable/retractable punching mechanism and the presetting application of assembly on the subject directly. The performing process includes taking test sample from subject for the test application and/or delivering pre-filled drugs into the subject.

3. If the assembly with the dressing portion 15, the assembly may be used as “a bandage like article” retain on the puncture site. The assembly may be stuck on said subject to protect the puncture site.

Further more, at the step 2: The performing of the extendable/retractable punching mechanism of assembly includes two sub-steps for some preset application. Said step 2-1 actuates said extension of needle to stay in the subject and said step 2-2 that withdrawn punching power actuates said retraction of needle. In the delivery application, it may need two sub-steps for the preset application configuration.

The invention assembly involves a method that combines with an associated device to perform the test or delivery task of the assembly. More importantly the assembly solves the problems of safety and convenience in performing process. The assembly may avoid any risks associate with the needle-stick, the contamination and cross-infection in the entire process. The assembly may have the follow useful features.

First, the assembly has a readable marker 23 (e.g. barcode, electric code) that provides information about assembly (shown on FIG. 5A for one kind of assembly). For example: when the assembly within the test application, the information includes an identification number of assembly, name of presetting application, aim of test component, and requirement of sample for test component, etc.

Second, the user easy uses the assembly via the associated device. The associated device 200 may be an integrated device with operating mechanism and/or measuring mechanism. For example, the lancing technology and measuring technology make up the associated device for glucose test monitoring.

According to measuring mechanism, the assembly is measured by either an electric analysis instrument or an optical instrument. The instruments may have an interface that connects to Internet source and allows input of relative information and output of applicable information.

According to operating mechanism, the operating system of device 200 is an operating tool for the assembly. The operating system has at least one couple-connector 211 and a perform-member 212. The couple-connector 211 uses to connect the connector-site 32 of the assembly and to load the assembly. The perform-member 212 presses or punches the needle to perform the assembly 100. The couple-connector 211 maybe moves out at loaded position to disconnect the connector-site 32 and to release assembly after the assembly preformed. The associated device 200 has power means to driver the perform-member 212. The power means may include one of spring power, an electric power, a mechanical power and a hydraulic power.

Third, the assembly provides a method and apparatus for a special associated device; it is called complete automated system or robot. The complete automated system or robot performs delivery application and/or test application for the healthcare industry.

The process comprises the follow steps:

a. automated loading a assembly to said complete automated system or robot.

b. automated applying said assembly to puncture area of subject.

c. automated entirely performing said extendable/retractable punching means and said preset application by complete automated system or robot; then catching and showing the result.

d. automated disconnecting assembly; the disconnected assembly may be continuing stuck on said subject.

What is claimed is:

1. A disposable automatic safety assembly comprising:
a housing pod;
an extendable/retractable punching means that comprises a punching needle and at least one recoverable pad disposed within said pod, said needle having a needle-body and a needle-head, and said needle-body being able to extend from said pod to puncture a subject and being able to retract within said pod after puncturing, said pad being able to change shape by pressure and recover without pressure; and
an application container within said pod, said application container housing applied component for a testing and/or delivery task;
an actuation mechanism that actuates said extension of said needle for performing testing and/or delivery task.

2. The assembly of claim 1, wherein said application container being configured for testing and/or delivery, the configuration of said application container includes at least one of the following: inside of said pad, inside of said needle, inside of said pod or house of said pod.

3. The assembly as defined in claim 1 wherein said pad is made of flexible materials with recovery ability, said flexible materials including any foamed materials (e.g. foam rubber), any soft materials (e.g. soft rubber).

4. The assembly as defined in claim 1 wherein said pad is a mixed pad, said mixed pad being made of different materials, said mixed pad including either mixed by different soft flexible materials or mixed by flexible materials and firm springy materials.

5. The assembly as defined in claim 1, wherein said pad having room or rooms, said room(s) of pad serving as a needle tunnel and/or storing space.

6. The assembly as defined in claim 5 said pad having a penetrable member to close its tunnel or room(s) at the subject end of said pad.

7. The assembly as defined in claim 1 wherein said needle is a lancet needle having a sharp edge or point for piercing the surface of subject.

8. The assembly as defined in claim 7 wherein said lancet needle having two sharps on said needle-body, said one sharp being used for piercing the surface of subject and said other sharp edge(s) being used for breaking said penetrable member of said pad.

9. The assembly as defined in claim 1 wherein said needle is a test probe to directly take sample from said subject for said test task of assembly.

10. The assembly as defined in claim 1 wherein said needle is a hollow hypodermic needle in communication with said application container, said hollow hypodermic needle including that has a hole of needle at side of said needle-body.

11. The assembly as defined in claim 1 wherein said needle is a double-ends hollow needle in communication with said application container, said communication to said application container following said extension of said needle.

12. The assembly as defined in claim 1, wherein comprising a ring member in said housing pod for guiding said extension of said needle.

13. The assembly as defined in claim 1, further comprising a dressing portion on a bottom-surface of said assembly, said dressing portion being stuck said assembly on the surface subject.

14. The assembly as defined in claim 1, wherein said test container comprises means housing a test component for testing task, said test component being reacted with sample taken from said subject, and said test component being configured for an electrochemical measuring or optical measuring.

15. The assembly as defined in claim 14, wherein said test container is slid in house of said pod with said extension of said needle, said slide test container being pushed to the surface of subject for catching test sample from the surface of subject directly.

16. The assembly as defined in claim 1, wherein said delivery container in said pad comprises means for storing injectable substance (e.g. drug) to be delivered into said subject.

17. The assembly as defined in claim 1, wherein said delivery container within said needle-head comprises means for storing injectable substance (e.g. drug) to be delivered into said subject.

18. The assembly according to claim 1 further comprising a changer-member with a blocker is used to lock said needle at initial position and/or to set a preset depth of puncturing for said assembly.

19. The assembly according to claim 1 further comprising a readable marker on said assembly that provides information on said assembly that information includes at least one of a type of application container disposed within said assembly, a type of needle disposed within said assembly, a type of drug to be delivered using said assembly, and a type of said testing within said assembly.

20. The assembly of claim 1, wherein comprising a loading connector-site on said pod that enables said assembly to be loaded and performed by an associated device.

21. The assembly of claim 20, said loading connector-site having electrode members for transfer electrical signal from said assembly to said associated device.

22. A method for using the assembly of claim 1:
applying said assembly to said subject;
performing said extendable/retractable punching means and said test and/or delivery task of said application container in one operating motion;
whereas said performing process includes taking sample from said subject for stored test component and/or delivering filled substance into said subject.

23. A method of an assembly within an extendable/retractable punching mechanism and an application container used by an associated device, the method comprising:
a. loading an assembly to an associated device;
b. applying said assembly to puncture area of subject;
c. performing said assembly by said associated device, said performing including extension of said needle for performance testing and/or delivery task of said application container, retraction of said needle within assembly, and catching performance result;
d. disconnecting said assembly, said disconnected assembly being either removed from said subject or stuck on said subject.

24. A disposable automatic safety assembly for healthcare comprising:
a housing pod;
an recoverable pad disposed within housing pod, said pad being able to change shape by pressure and recover without pressure;
a punching needle disposed within housing pod, said needle having a needle-body and a needle-head, and said needle-body being able to extend from said housing pod to puncture a subject and being able to retract within said housing pod after puncturing.
